(19) United States
(12) Patent Application Publication
(10) Pub. No.: US 2013/0071471 A1
(43) Pub. Date: Mar. 21, 2013

(54) PHARMACEUTICAL COMPOSITION FOR TREATING ATOPIC DERMATITIS

(71) Applicant: Hanall Biopharma Co., LTD., Daejeon (KR)

(72) Inventors: Sung Soo JUN, Gyeonggi-do (KR); Soon Im LEE, Seoul (KR)

(73) Assignee: HANALL BIOPHARMA CO., LTD., Daejeon (KR)

(21) Appl. No.: 13/679,056

(22) Filed: Nov. 16, 2012

Related U.S. Application Data
(63) Continuation-in-part of application No. 13/016,682, filed on Jan. 28, 2011, now abandoned.
Application No. 13/016,682 is a continuation of application No. 11/996,574, filed on Jan. 23, 2008, now abandoned.

(30) Foreign Application Priority Data
Dec. 9, 2005 (KR) 10-2005-0120648

(51) Int. Cl. A61K 31/714

(52) U.S. Cl. CPC A61K 31/714
USPC 424/450; 536/26.4; 514/47; 424/94.1

(57) ABSTRACT

A method of treating mild atopic dermatitis patients by locally administering an external composition containing adenosylcobalamin as an active ingredient is provided. The composition can effectively treat mild atopic dermatitis patients without side effects.

### CROSS-REFERENCE TO RELATED APPLICATIONS

This application is a continuation-in-part of U.S. patent application Ser. No. 13/016,682 filed Jan. 8, 2011 (pending), which is a continuation of U.S. patent application Ser. No. 11/996,574 filed Jan. 23, 2008 entitled “Compositions for External Application, Containing Adenosylcobalamin for Improvement of Skin Diseases” (abandoned), which application is a 371 of PCT/KR2006/004233 filed on Oct. 18, 2006, published on Jun. 14, 2007 under publication number WO 2007/066889 A1 which claims priority benefits from South Korean Patent Application Number 10-2005-0120648 filed Dec. 9, 2005, the disclosures of which are hereby incorporated by reference.

### TECHNICAL FIELD

The present invention provides a method of treating a mild atopic dermatitis patient using an external composition containing adenosylcobalamin as an active ingredient.

### BACKGROUND ART

Atopic dermatitis is chronically relapsing dermatitis having severe itchiness. In Korea, it is difficult to estimate an exact incidence of atopic dermatitis, but its prevalence is consistently on the rise. It is reported that the prevalence is approximately 10 to 20% and it varies depending on region, age, sex, and sociocultural characteristics.

There are various medicines which can be locally administered to atopic dermatitis lesions. Today, the most frequently used therapeutic agent to treat atopic dermatitis is steroids. An overdose of steroids causes depletion of steroid binding receptors, thereby leading to tolerance, and thus forcing atopic dermatitis patients to apply medicine more frequently or use a medicine having a stronger effect. As patients use more steroids, the steroids are converted into peroxides on their skin and gradually aggravate the skin disease. Steroids are more easily absorbed through thinner skin. Particularly, since steroids are more easily absorbed in children, the use of steroids is restricted due to their side effects.

Besides steroids, immunoregulators and antihistamines are used. However, immunoregulators have a higher chance of inducing skin cancer, although it has fewer side effects than steroids. In addition, the use of antihistamines for alleviating pruritus improves pruritus, but it leads to a vicious cycle of pruritus-scratching-atopic dermatitis-pruritus and becomes difficult to treat. Moreover, the effects of antihistamines on atopic dermatitis are not yet proven. Accordingly, there is an urgent need to develop a medicine that is safe and effective for treating atopic dermatitis.

To this end, the present applicant filed Korean Patent Application No. 2005-0120648 (registered as Korean Patent No. 0658436) relating to a composition for treating skin diseases containing adenosylcobalamin (coenzyme B12). The external composition containing adenosylcobalamin is an external drug for treating skin diseases, which is prepared by improving disadvantages of the conventional cobalamine (vitamin B12) formulation, including difficulty in expecting a quick medical effect and very low bioavailability since 90% or more of a dose is excreted due to a very large molecular weight. The external drug contains adenosylcobalamin constituting a coenzyme of cobalamine as an active ingredient and facilitates penetration of an epidermal barrier. Particularly, the adenosylcobalamin-containing external composition has an excellent effect on atopic dermatitis.

### DISCLOSURE

### Technical Problem

In the present invention, it has been confirmed that the external composition containing adenosylcobalamin as an active ingredient is highly effective in treating patients suffering from mild atopic dermatitis.

Therefore, the present invention provides a method of treating a mild atopic dermatitis patient by locally administering an external composition containing adenosylcobalamin as an active ingredient and an external composition containing adenosylcobalamin as an active ingredient to treat mild atopic dermatitis.

In the present invention, it has also been confirmed that the external composition containing adenosylcobalamin as an active ingredient is more highly effective in treating female patients suffering from mild atopic dermatitis.

Accordingly, the present invention also provides a method of treating a female mild atopic dermatitis patient by locally administering an external composition containing adenosylcobalamin as an active ingredient and an external composition containing adenosylcobalamin as an active ingredient to treat mild atopic dermatitis in females.

### Technical Solution

One aspect of the present invention provides a method of treating a mild atopic dermatitis patient by locally administering the external composition containing adenosylcobalamin as an active ingredient.

Atopic dermatitis patient refers to a patient diagnosed with atopic dermatitis, and atopic dermatitis is usually diagnosed based on clinical symptoms since there are no specific methods or examination methods. In one example, atopic dermatitis may be diagnosed according to diagnostic criteria for atopic dermatitis of Hanifin & Rajka.

The severity of atopic dermatitis may be expressed as mild, moderate and severe according to the degree of the symptoms. In the present invention, change in the Eczema Area and Severity Index (EASI) score was determined as the primary parameter of efficacy evaluation. When the severity of atopic dermatitis is rated using the EASI score, mild symptoms are given an EASI score of 10 or less, moderate symptoms are given an EASI score of 20 or less but more than 10, and severe symptoms are given an EASI score of more than 20. In the present invention, mild atopic dermatitis refers to atopic dermatitis having an EASI score of 10 or less but more than 2. When the EASI score is less than 2, symptoms of atopic dermatitis are insignificant, and obvious effects of the external composition of the present invention cannot be measured. Thus, cases having an EASI score of less than 2 were excluded. As a result, a mild atopic dermatitis patient of the present invention refers to an atopic dermatitis patient having an EASI score of 10 or less but more than 2 when atopic dermatitis was diagnosed.

The external composition containing adenosylcobalamin as an active ingredient and a method of preparing the same are disclosed in Korean Patent Application No. 2005-0120648, filed by the present applicant, and the content of which is incorporated herein by reference in its entirety. Preferably, the external composition of the present invention is a gel-type external composition in which adenosylcobalamin is encapsulated with a liposome including a phospholipid and cholesterol. When necessary, a surfactant containing an alkyl group having 8 to 16 carbon atoms may be further included to enhance skin absorbability and skin compatibility. The content of the adenosylcobalamin may be, but is not limited to, 0.01 to 7 wt % based on the total composition of the external composition. In one embodiment, a content of the adenosylcobalamin may be 0.01 to 1 wt % based on the total composition of the external composition.

“Administration” used herein refers to a thin application of the external composition of the present invention to an atopic dermatitis lesion. As one of the application method of the present invention, a thin layer of the external composition may be applied twice a day at intervals of approximately 8 to 14 hours. In one embodiment, the present invention includes continuously administering the external composition containing adenosylcobalamin as an active ingredient for 4 weeks or more, 6 weeks or more or 8 weeks or more.

In another embodiment, the present invention provides a method of treating a female mild atopic dermatitis patient by locally administering the external composition containing adenosylcobalamin as an active ingredient to the female mild atopic dermatitis patient.

In the present invention, the female patient has had her first period. In one embodiment, the age of the female patient is not limited, but is generally at least 12 years old. It is known that atopic dermatitis occurs more frequently in females after adolescence (Osman et al, Arch Dis Child 2003; 88; 587-590), but there are no case studies to confirm a difference in treatment effect between female and male patients.

In still another embodiment, the present invention provides an external composition containing adenosylcobalamin as an active ingredient to treat mild atopic dermatitis.

In yet another embodiment, the present invention provides an external composition containing adenosylcobalamin as an active ingredient to treat mild atopic dermatitis in females.

To confirm a treatment effect of the external composition containing adenosylcobalamin on atopic dermatitis patients, double-blind, randomized, vehicle-controlled, and parallel study thereof were performed for 8 weeks.

0.07% and 0.30% adenosylcobalamin external gel test drugs and a gel control drug containing 0.3% nanoliposome excluding adenosylcobalamin were administered to a total of 125 mild atopic dermatitis patients (including 28 patients having an EASI score of 2 or less and 97 patients having an EASI score of 10 or less but more than 2) and a total of 37 patients of moderate atopic dermatitis (having EASI score of more than 10). As a result, there were significant effects in all test categories including change in EASI scores, percentage change in EASI scores, IGA treatment success rates, change in IGA scores, pruritus, insomnia, and evaluation of quality of life in the mild atopic dermatitis patients having an EASI score of 10 or less but more than 2.

In addition, in female mild atopic dermatitis patients having an EASI score of 10 or less but more than 2, there were particularly excellent effects in all of the evaluation categories mentioned above, compared with male patients.

### Advantageous Effects

As described above, the present invention can safely and effectively treat mild atopic dermatitis patients using an external composition containing adenosylcobalamin as an active ingredient.

### DESCRIPTION OF DRAWINGS

FIG. 1 is a graph showing change in EASI score of atopic dermatitis patients having an EASI score of (i) 2 or less, (ii) 10 or less but more than 2, and (iii) more than 10, measured according to a clinical experiment of the present invention;

FIG. 2 is a graph showing percentage change in EASI score of the atopic dermatitis patients having an EASI score of (i) 2 or less, (ii) 10 or less but more than 2, and (iii) more than 10, measured according to a clinical experiment of the present invention;

FIG. 3 is a graph showing an IGA treatment success rate of atopic dermatitis patients having an EASI score of (i) 2 or less, (ii) 10 or less but more than 2, and (iii) more than 10, measured according to a clinical experiment of the present invention;

FIG. 4 is a graph showing change in IGA score of atopic dermatitis patients having an EASI score of (i) 2 or less, (ii) 10 or less but more than 2, and (iii) more than 10, measured according to a clinical experiment of the present invention;

FIG. 5 is a graph showing change in degree of pruritus of atopic dermatitis patients having an EASI score of (i) 2 or less, (ii) 10 or less but more than 2, and (iii) more than 10, measured according to a clinical experiment of the present invention;

FIG. 6 is a graph showing change in degree of insomnia of atopic dermatitis patients having an EASI score of (i) 2 or less, (ii) 10 or less but more than 2, and (iii) more than 10, measured according to a clinical experiment of the present invention;

FIG. 7 is a graph showing change in quality of life of atopic dermatitis patients having an EASI score of (i) 2 or less, (ii) 10 or less but more than 2, and (iii) more than 10, measured according to a clinical experiment of the present invention;

FIG. 8 is a graph showing change in EASI scores of male and female atopic dermatitis patients having an EASI score of 10 or less but more than 2, measured according to a clinical experiment of the present invention;

FIG. 9 is a graph showing percentage change in EASI scores of male and female atopic dermatitis patients having an EASI score of 10 or less but more than 2, measured according to a clinical experiment of the present invention;

FIG. 10 is a graph showing an IGA treatment success rates of male and female atopic dermatitis patients having an EASI score of 10 or less but more than 2, measured according to a clinical experiment of the present invention;

FIG. 11 is a graph showing change in IGA scores of male and female atopic dermatitis patients having an EASI score of 10 or less but more than 2, measured according to a clinical experiment of the present invention;

FIG. 12 is a graph showing change in degree of pruritus of male and female atopic dermatitis patients having an EASI score of 10 or less but more than 2, measured according to a clinical experiment of the present invention;

FIG. 13 is a graph showing change in degree of insomnia of male and female atopic dermatitis patients having an EASI score of 10 or less but more than 2, measured according to a clinical experiment of the present invention; and

FIG. 14 is a graph showing change in degree of quality of life of male and female atopic dermatitis patients having an EASI score of 10 or less but more than 2, measured according to a clinical experiment of the present invention.

### MODE FOR INVENTION

Hereinafter, the exemplary Preparation Examples and Examples are provided to aid in understanding the present invention, but they are merely provided to describe the present invention, not to limit the scope of the present invention.

### Preparation Example 1

### Preparation of External Gel Containing 0.07% Adenosylcobalamin (Test Drug 1)

Preparation of an external gel containing adenosylcobalamin of the present invention was performed by the following method under a light-shielding condition. After propyleneglycol was put into a paddle mixer tank, cholesterol and stearoyl phosphatidyl choline were added in a ratio of 1:3, and then completely dissolved with stirring. 35 g of adenosylcobalamin was precisely weighed and added to 3,500 g of distilled water in a separate container to completely dissolve the adenosylcobalmaine. Afterward, the resulting solution was put into the paddle mixer tank. The solution was extruded using a nano disperser (Ilsin Autoclave) until particles having a size of 100 to 200 nm were obtained, and thereby a liposome was prepared.

To prepare a gel-base material, distilled water and a carbomer were stirred in a homo mixer tank to completely dissolve the carbomer. Here, glycerin and sodium acetate hydrate were added and completely dissolved, and then mixed with methyl paraoxybenzoate and propyl paraoxybenzoate using a homo mixer until the mixture was homogenized. After the mixing was completed, trolamine was added to adjust the pH to 6.5 to 7.5, and thus the gel-base material was obtained.

The liposome previously prepared was put into the mixer tank containing the gel-base material and stirred for 30 minutes, thereby obtaining a total of 50 kg of Test Drug 1 containing 0.07% adenosylcobalamin.

### Preparation Example 2

### Preparation of External Gel Containing 0.3% Adenosylcobalamin (Test Drug 2)

2.50 kg of Test Drug 2 containing 0.3% adenosylcobalamin was obtained by the same method as described in Preparation Example 1, except that 150 g of adenosylcobalamin was added to 15,000 g of distilled water.

### Preparation Example 3

### Preparation of Adenosylcobalamin-free Vehicle Gel (Control Drug)

As a Control Drug of the clinical experiment, a nanoliposome gel not containing adenosylcobalamin as a main ingredient of the Test Drug was prepared. The object of the experiment was to confirm the effect of pure adenosylcobalamin only with respect to atopic dermatitis, and the Control Drug was designed by excluding only adenosylcobalamine from a Test Drug. The clinical experiment was performed for a somewhat medium-long period of 8 weeks. Steroids and immunosuppressants have safety problems, and they are secondary medicine or short-term and intermittent medicine. Therefore, steroids and immunosuppressants are inappropriate to compare with the external composition containing adenosylcobalamin. For this reason, the Control Drug was designed as described above.

In addition, since the Test Drug was reddish due to characteristics of the main ingredient, a vehicle was prepared by adding similar red pigments to the Control Drug for double-blind, and the pigments were Red Pigments 227 and 40 specified in “Guidelines for Classifying Tarr Colors for Medicines and Medical Supplies, Sanitary Supplies and Cosmetics, and Standards and Test Methods for the Same.”

### Example 1

### Clinical Protocols

### Example 1-1

### Criteria for Screening Clinical Subjects

Male and female patients at ages 12 to 18, who were diagnosed with atopic dermatitis according to the Hanifin & Rajka method, were selected, and then subjects corresponding to mild and moderate atopic dermatitis were selected therefrom based on EASI score according to the determination by a researcher. In addition, exemption criteria were established. Subjects meeting the exemption criteria included subjects treated with drugs who required a washout period and subjects who were allergic to vitamin B12 were excluded, and therefore, 162 patients were finally screened.

### Example 1-2

### Experimental Method

Random numbers were allocated to respective subjects at week 0 (Visit 2) of the clinical experiment. Group assignment was performed with respect to Test Drug 1 (HL-009 0.07%), Test Drug 2 (HL-009 0.30%) and the Control Drug in a ratio of 1:1:1, and the experiment was finally performed on a total of 162 subjects: Test Drug 1 group was assigned 52 subjects, Test Drug 2 group was assigned 54 subjects, and the control group was assigned 56 subjects.

The experiment period per subject was 10 weeks in total, and the experiment was performed in a total of 7 visits (screening, 0-week, 2-week, 4-week, 6-week, 8-week and 10-week) if the screening was performed. When 8-week treatment including a screening period of less than 4 weeks according to the washout period was completed, after 2 weeks of the completion of the treatment, safety was confirmed by a follow-up visit. If a washout period was not necessary, visits 1 and 2 were simultaneously performed.

After the random allocation of drugs, the subjects were allowed to visit once every two weeks during the treatment to be prescribed drugs for the clinical experiment for two weeks at visits 2, 3, 4 and 5 and to return empty tubes of the drugs for the clinical experiment previously prescribed and the drugs for the clinical experiment remaining after use at visits 3, 4, 5 and 6.

### Example 1-3

### Dose, Period and Administration Method

A thin layer of Test Drug 1, Test Drug 2 and the Control Drug were applied twice a day at intervals of approximately 8 to 14 hours for 8 weeks in the following amount in consideration of a region and an area of atopic dermatitis. The predetermined amount was 0.07 g per 1 cm of gel squeezed out of the tube for the clinical experiment (diameter of its entrance: 1 mm), and then the above amount of the gel was applied to cover an approximately 3.3 cm by 3.3 cm area of the body surface.

Moisturizers containing no steroids (e.g., a Neutrogena® Moisturizer, and a Johnson & Johnson Moisturizer) were suitably applied to the region of atopic dermatitis twice a day after the baseline for 8 weeks. Moisturizers were applied at least 30 minutes after application of the drug for the clinical experiment.

### Example 1-4

### Evaluation Method

In an outpatient service, researchers evaluated subjects' atopic dermatitis (EASI and IGA), safety, prutitus, insomnia and quality of life.

### Example 1-5

### Parameters for Evaluation of Efficacy and Safety

In the clinical experiment, the change in EASI score was set as a primary parameter of efficacy evaluation. Evaluations were performed using a secondary parameter of efficacy evaluation including percentage change in EASI score, an IGA treatment success rate, change in IGA score, degree of pruritus and insomnia, and change in quality of life.

1) Change in EASI Score

An area of the body surface was roughly divided into 4 parts: a head and neck part, an upper limb part, a torso part and a lower limb part. At each part of the body, degree of acute/chronic inflammation in atopic dermatitis such as erythema, induration/population, excoriation and lichenification were observed, and their severities were expressed as a number ranging from 0 to 3. In addition, an area of the body surface exhibiting the above-mentioned symptoms in the four parts was measured and expressed as a number. The severities were rated from 0, which means that there are no symptoms, to 6, which means that the symptoms occur over 90 to 100% of the area of the body surface. Moreover, each body part was given a weight according to age. The group of age 8 or more was given different weights depending on the parts of the body, for example, 10% at the head and neck part, 20% at the upper limb part, 30% at the torso part, and 40% at the lower limb part. The EASI score was expressed by summing the values obtained by multiplying the rated area of the body surface and symptoms by the different respective percentages depending on age, to rate the severity from 0 to 72. Using the same method, the scores were measured before and after administration, and then change in the EASI score was evaluated.

2) Percentage Change in EASI Score

Percentage change in EASI score was evaluated by converting the change in EASI score after administration, compared with those before administration, into a percentage.

3) IGA Treatment Success Rate and Change in IGA Score

IGA scores are evaluated in 6 grades ranging from 0 to 5 according to criteria listed in Table 1 (IGA scores).

TABLE 1
IGA Scores

| Score | Symptoms |
|---|---|
| 0 = Clear | No atopic inflammation |
| 1 = Almost clear | A little detectable erythema or |
| | papulation/infiltration |
| 2 = Mild | Mild erythema or papulation/infiltration |
| 3 = Moderate | Moderate erythema or papulation/infiltration |
| 4 = Severe | Severe erythema or |
| | papulation/infiltration |
| 5 = Very severe | Severe exudative erythema or |
| | papulation/infiltration with boils |

The IGA treatment success rate was confirmed by considering treatment to be successful if an IGA score of a subject was 0 or 1 when the experiment was completed.

The change in IGA score was used to evaluate variance in IGA scores after administration compared to before administration.

4) Evaluation of Pruritus and Insomnia

Pruritus/insomnia of subjects was evaluated using a 10 cm visual analog scale (VAS) to grade the degree of pruritus/insomnia from 0, which indicates that there is no pruritus/insomnia, to 10, which indicates that pruritus/insomnia is the most severe. Accordingly, the change in degree of pruritus/insomnia after administration compared to before administration was evaluated.

5) Evaluation of Quality of Life (QOL)

Evaluation of quality of life was performed with a total of 10 questions relating to the symptoms and the patients' perceptions, spare time and leisure, school life or holidays, personal relationships, sleep and treatment. A subject rated each question as “very much (3 points), considerably (2 points), a little (1 point) and not at all (0),” and then the points were summed together to evaluate the change in score after administration compared to before administration.

### Example 2

### Comparison of Effects per EASI Score Group (Comparison of Effects for Patients of Mild vs. Moderate Atopic Dermatitis)

Results obtained by confirming effects with respect to atopic dermatitis patients having EASI score of 2 or less, 10 or less but more than 2, and more than 10 are as follows. The effects were confirmed according to the change in EASI score (Table 2), the percentage change in EASI score (Table 3), the IGA treatment success rate (Table 4), the change in IGA score (Table 5), the degree of pruritus (Table 6), the degree of insomnia (Table 7), and the change in quality of life (Table 8).

TABLE 2
Change in EASI Score

| Range of Initial | | Period of Treatment | | | |
|---|---|---|---|---|---|
| EASI Score | Test Group | 2 weeks | 4 weeks | 6 weeks | 8 weeks |
| 2 or less | 0.07% (n = 9) | 0.11 | 0.20 | 0.11 | 0.23 |
| | 0.3% (n = 8) | 0.06 | 0.48 | 0.38 | 0.23 |
| | Control Drug | 0.41 | 0.58 | 0.77 | 0.86 |
| | (n = 11) | | | | |
| 10 or less but | 0.07% (N = 30) | 0.42 | 1.45 | 1.78 | 2.17 |
| more than 2 | 0.3% (N = 32) | 0.38 | 1.03 | 1.54 | 2.09 |
| | Control Drug | 0.74 | 0.80 | 1.19 | 1.79 |
| | (N = 34) | | | | |
| more than 10 | 0.07% (n = 12) | 3.23 | 4.98 | 5.71 | 6.84 |
| | 0.3% (n = 14) | 2.29 | 1.31 | 2.39 | 3.45 |
| | Control Drug | 2.95 | 4.41 | 7.22 | 8.05 |
| | (n = 11) | | | | |

TABLE 3
Percentage Change in EASI Score

| Range of Initial | | Period of Treatment | | | |
|---|---|---|---|---|---|
| EASI Score | Test Group | 2 weeks | 4 weeks | 6 weeks | 8 weeks |
| 2 or less | 0.07% (n = 9) | 8.10 | 19.88 | 3.39 | 10.90 |
| | 0.3% (n = 8) | 8.15 | 23.94 | 16.24 | 4.26 |
| | Control Drug | 28.32 | 40.41 | 52.88 | 60.62 |
| | (n = 11) | | | | |
| 10 or less but | 0.07% (N = 30) | 8.51 | 26.01 | 32.00 | 40.01 |
| more than 2 | 0.3% (N = 32) | 8.61 | 20.22 | 27.18 | 34.57 |
| | Control Drug | 14.61 | 16.28 | 22.95 | 33.64 |
| | (N = 34) | | | | |
| more than 10 | 0.07% (n = 12) | 21.52 | 33.03 | 36.97 | 44.71 |
| | 0.3% (n = 14) | 18.97 | 11.07 | 23.94 | 26.91 |
| | Control Drug | 20.05 | 29.05 | 49.75 | 55.27 |
| | (n = 11) | | | | |

TABLE 4
IGA Treatment Success Rate

| Range of Initial | | Period of Treatment | | | |
|---|---|---|---|---|---|
| EASI Score | Test Group | 2 weeks | 4 weeks | 6 weeks | 8 weeks |
| 2 or less | 0.07% (n = 9) | 11.1 | 22.2 | 33.3 | 66.7 |
| | 0.3% (n = 8) | 12.5 | 25.0 | 25.0 | 25.0 |
| | Control Drug | 27.3 | 45.5 | 63.6 | 81.8 |
| | (n = 11) | | | | |
| 10 or less but | 0.07% (N = 30) | 3.3 | 10.0 | 13.3 | 36.7 |
| more than 2 | 0.3% (N = 32) | 3.1 | 6.3 | 12.5 | 25.0 |
| | Control Drug | 8.8 | 9.1 | 18.8 | 23.5 |
| | (N = 34) | | | | |
| more than 10 | 0.07% (n = 12) | 0.0 | 0.0 | 8.3 | 16.7 |
| | 0.3% (n = 14) | 0.0 | 0.0 | 0.0 | 7.1 |
| | Control Drug | 0.00 | 0.00 | 27.3 | 18.2 |
| | (n = 11) | | | | |

TABLE 5
Change in IGA Score

| Range of Initial | | Period of Treatment | | | |
|---|---|---|---|---|---|
| EASI Score | Test Group | 2 weeks | 4 weeks | 6 weeks | 8 weeks |
| 2 or less | 0.07% (n = 9) | 0.18 | 0.55 | 0.55 | 1.27 |
| | 0.3% (n = 8) | 0.00 | 0.00 | 0.00 | 0.13 |
| | Control Drug | 0.27 | 0.45 | 0.73 | 1.00 |
| | (n = 11) | | | | |
| 10 or less but | 0.07% (N = 30) | 0.20 | 0.27 | 0.43 | 0.70 |
| more than 2 | 0.3% (N = 32) | 0.13 | 0.25 | 0.28 | 0.59 |
| | Control Drug | 0.06 | 0.12 | 0.26 | 0.41 |
| | (N = 34) | | | | |
| more than 10 | 0.07% (n = 12) | 0.17 | 0.33 | 0.58 | 0.58 |
| | 0.3% (n = 14) | 0.21 | 0.07 | 0.21 | 0.29 |
| | Control Drug | 0.18 | 0.45 | 0.55 | 0.55 |
| | (n = 11) | | | | |

TABLE 6
Evaluation of Degree of Pruritus

| Range of Initial | | Period of Treatment | | | |
|---|---|---|---|---|---|
| EASI Score | Test Group | 2 weeks | 4 weeks | 6 weeks | 8 weeks |
| 2 or less | 0.07% (n = 8) | 0.89 | 1.61 | 0.43 | 1.76 |
| | 0.3% (n = 8) | 0.38 | −0.01 | 0.34 | 0.85 |
| | Control Drug | 0.50 | 1.49 | 2.09 | 2.46 |
| | (n = 11) | | | | |
| 10 or less but | 0.07% (N = 30) | 1.23 | 2.13 | 2.13 | 2.60 |
| more than 2 | 0.3% (N = 32) | 0.93 | 1.52 | 1.84 | 2.67 |
| | Control Drug | 0.95 | 0.92 | 1.28 | 1.65 |
| | (N = 34) | | | | |
| more than 10 | 0.07% (n = 12) | 0.23 | 0.49 | 1.32 | 1.26 |
| | 0.3% (n = 13) | 0.68 | 1.33 | 0.99 | 1.24 |
| | Control Drug | 1.88 | 2.78 | 2.21 | 3.01 |
| | (n = 11) | | | | |

TABLE 7
Evaluation of Degree of Insomnia

| Range of Initial | | Period of Treatment | | | |
|---|---|---|---|---|---|
| EASI Score | Test Group | 2 weeks | 4 weeks | 6 weeks | 8 weeks |
| 2 or less | 0.07% (n = 8) | 0.11 | 0.49 | −0.29 | −0.03 |
| | 0.3% (n = 8) | −0.08 | −0.10 | −0.10 | −0.14 |
| | Control Drug | −0.10 | 0.05 | 0.27 | 0.44 |
| | (n = 11) | | | | |
| 10 or less but | 0.07% (N = 30) | 0.35 | 0.55 | 0.99 | 1.58 |
| more than 2 | 0.3% (N = 32) | 0.73 | 1.05 | 1.50 | 1.92 |
| | Control Drug | 0.53 | 0.56 | 0.71 | 0.96 |
| | (N = 34) | | | | |
| more than 10 | 0.07% (n = 12) | 0.37 | 1.57 | 1.99 | 2.08 |
| | 0.3% (n = 13) | 0.34 | 1.38 | 1.30 | 1.58 |
| | Control Drug | 1.06 | 1.64 | 1.43 | 1.61 |
| | (n = 11) | | | | |

TABLE 8
Evaluation of Change in Quality of Life

| Range of Initial | | Period of Treatment | | | |
|---|---|---|---|---|---|
| EASI Score | Test Group | 2 weeks | 4 weeks | 6 weeks | 8 weeks |
| 2 or less | 0.07% (n = 9) | 0.33 | 1.00 | 1.00 | 2.11 |
| | 0.3% (n = 8) | −0.63 | −1.00 | −1.13 | 0.00 |
| | Control Drug | 2.18 | 1.82 | 2.27 | 3.45 |
| | (n = 11) | | | | |
| 10 or less but | 0.07% (N = 30) | 1.58 | 3.10 | 3.68 | 4.68 |
| more than 2 | 0.3% (N = 32) | 1.25 | 2.53 | 3.38 | 4.06 |
| | Control Drug | 1.06 | 1.35 | 1.53 | 2.06 |
| | (N = 34) | | | | |
| more than 10 | 0.07% (n = 12) | 4.58 | 3.83 | 5.92 | 6.83 |
| | 0.3% (n = 13) | 0.92 | 2.15 | 1.46 | 1.15 |
| | Control Drug | 2.00 | 3.27 | 2.82 | 3.64 |
| | (n = 11) | | | | |

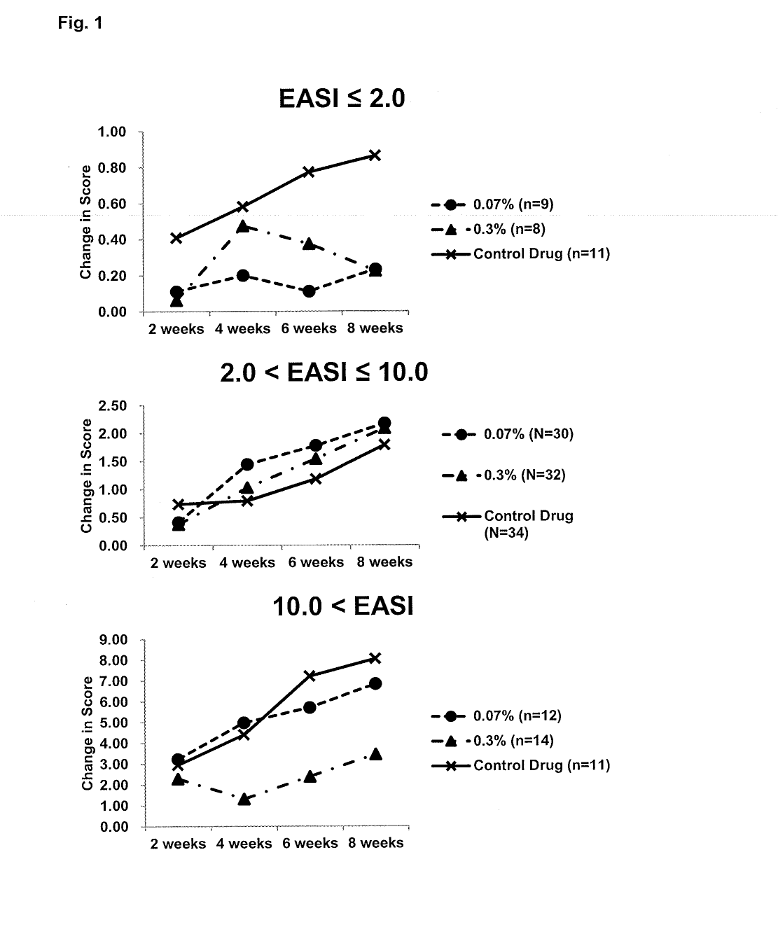

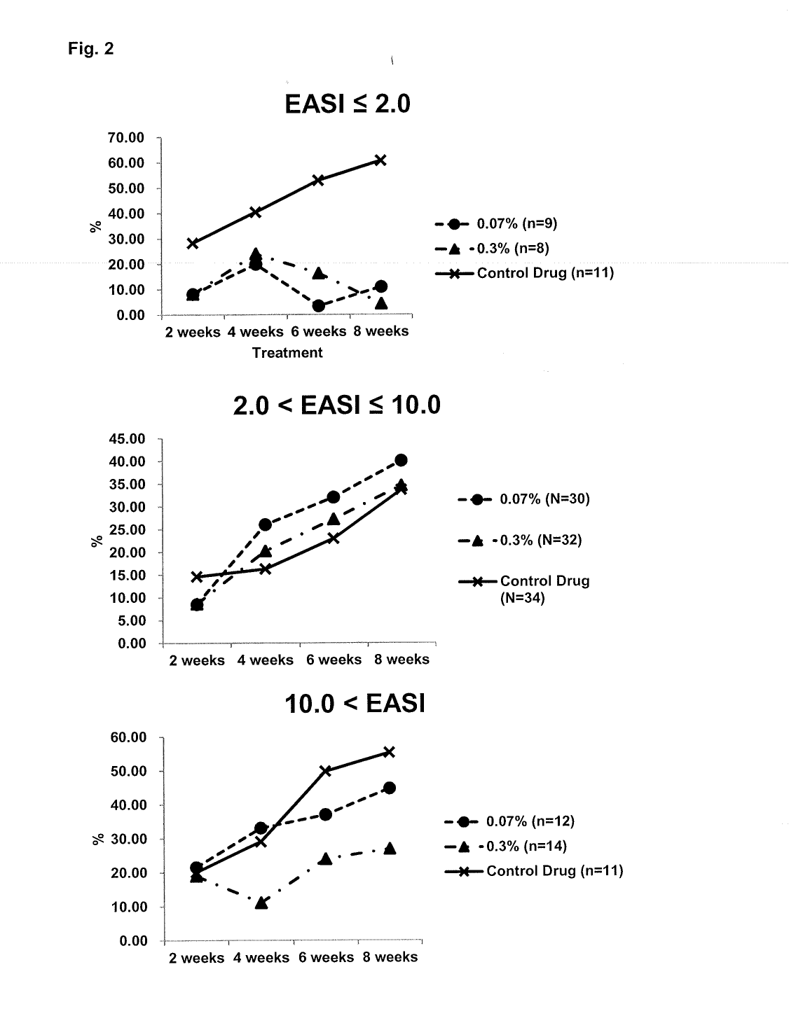

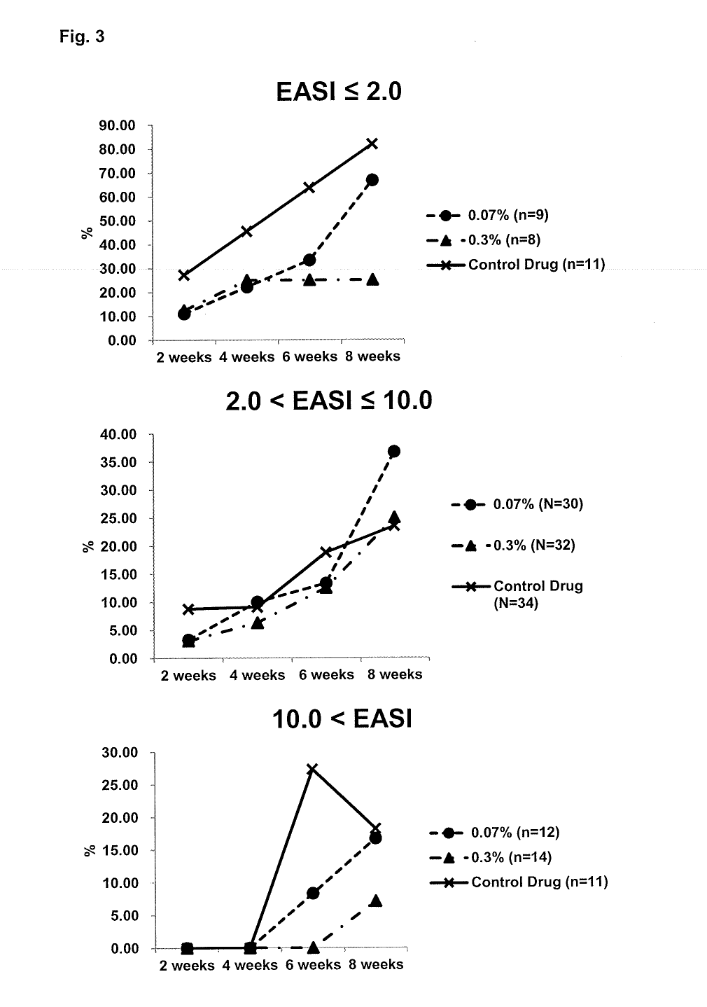

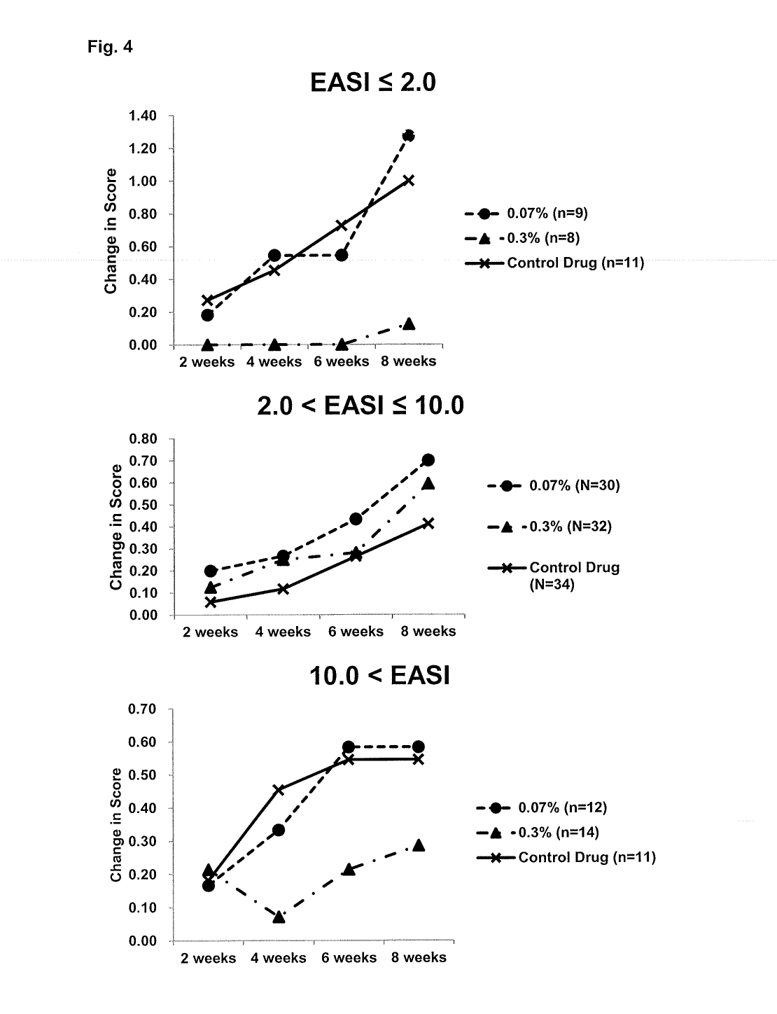

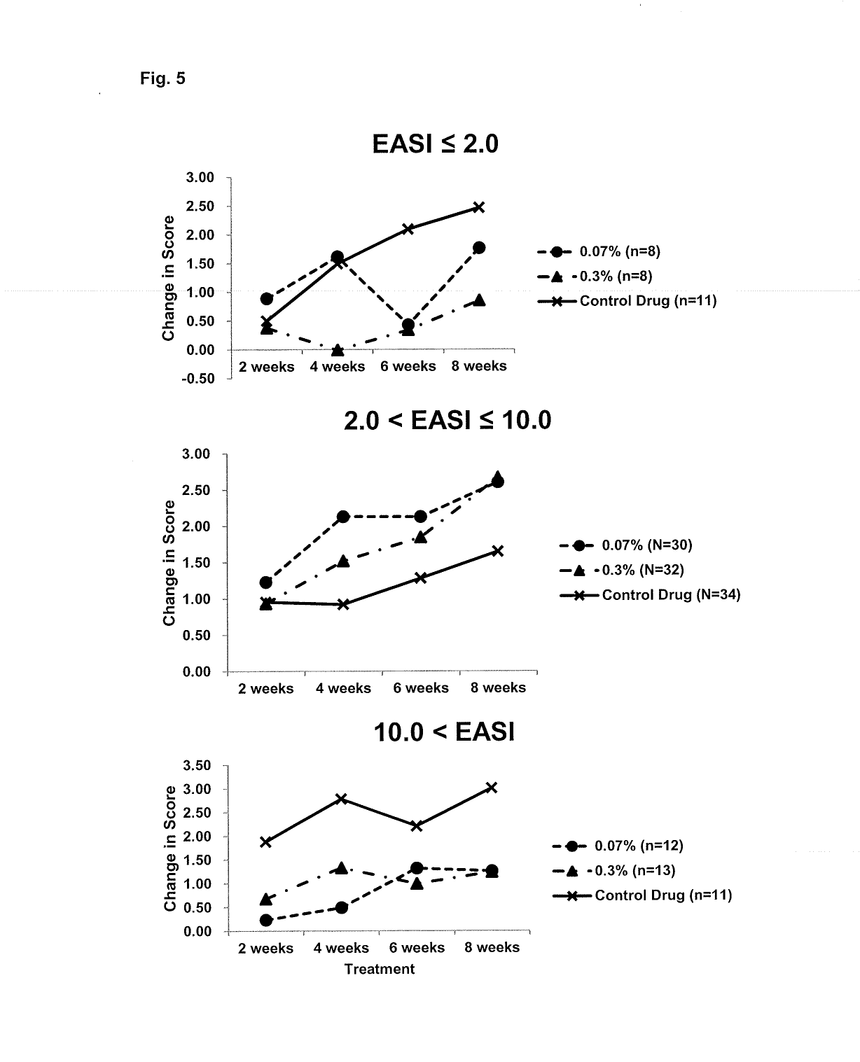

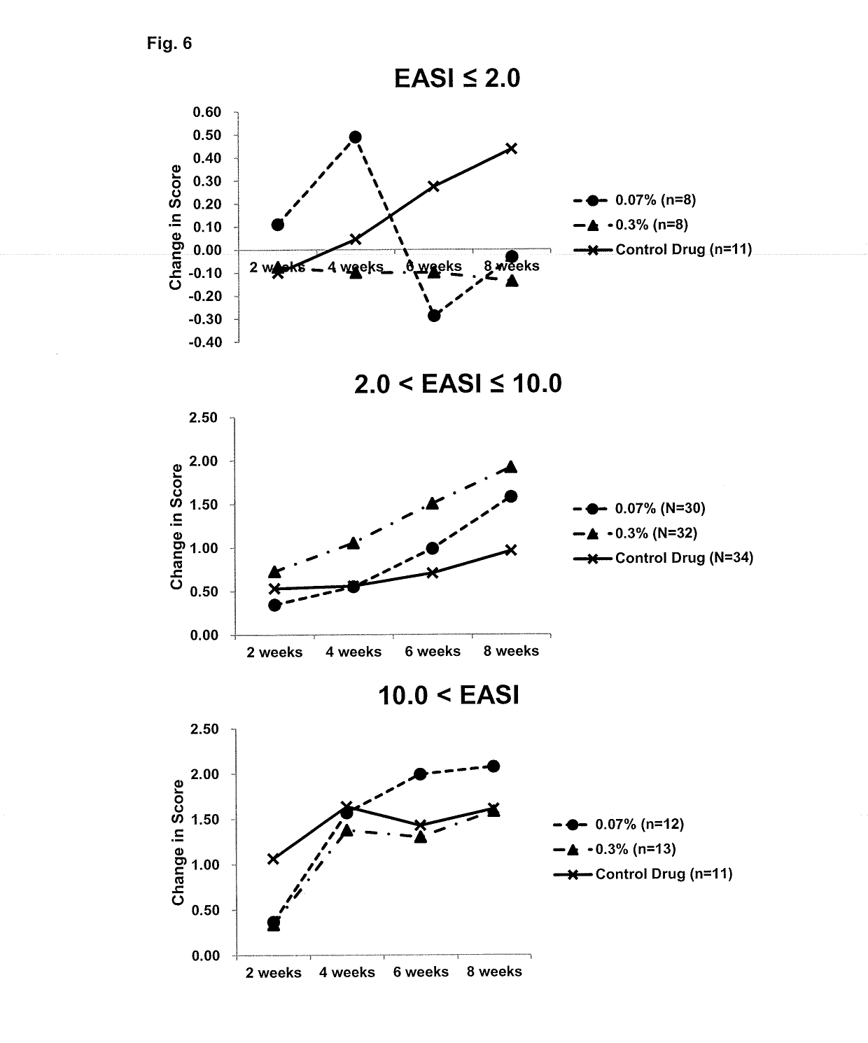

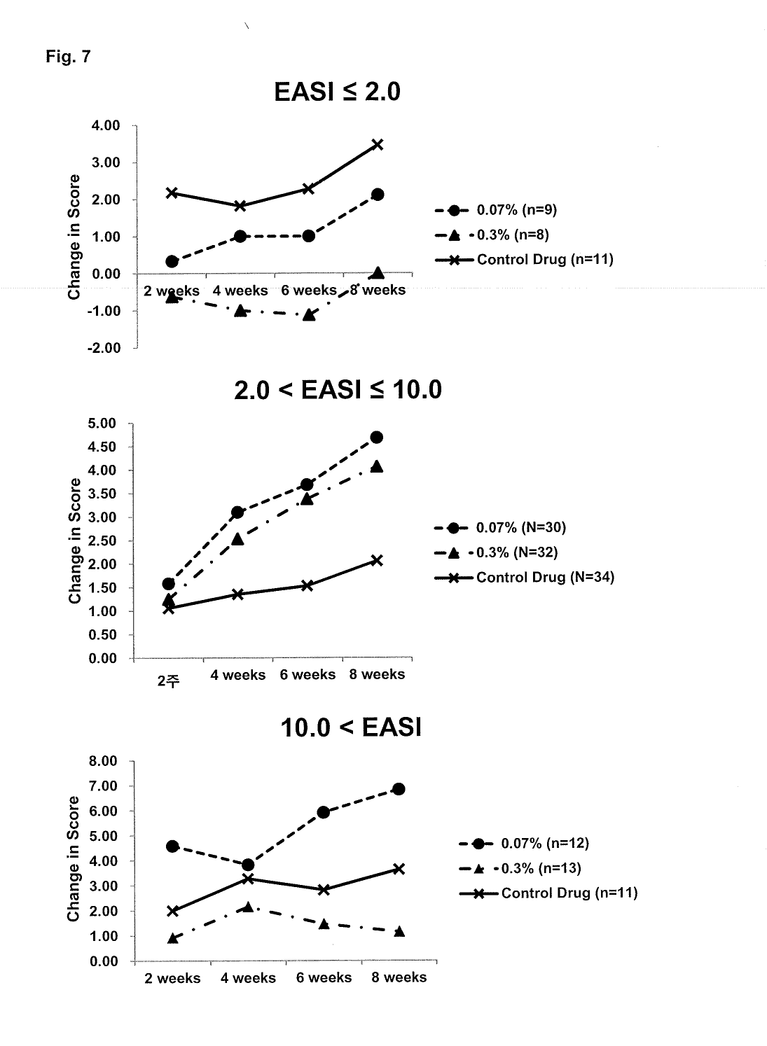

As shown in Tables 2 to 8 and FIGS. 1 to 7, it can be seen that in mild atopic dermatitis patients having EASI score of 10 or less but more than 2, adenosylcobalamin was a more effective treatment on atopic dermatitis than in atopic dermatitis patients having EASI score of 2 or less and atopic dermatitis patients having EASI score of more than 10, in all test categories.

### Example 3

### Comparison of Effects on Male and Female Atopic Dermatitis Patients Having EASI Score of 10 or Less but More Than 2

Results obtained by comparing effects on male and female atopic dermatitis patients having EASI score of 10 or less but more than 2 are as follows. The effects were confirmed based on the change in EASI score (Table 9), the percentage change in EASI score (Table 10), the IGA treatment success rate (Table 11), the change in IGA score (Table 12), the degree of pruritus (Table 13), the degree of insomnia (Table 14), and the change in quality of life (Table 15).

TABLE 9
Change in EASI Score

| Male/Female | | | | | |
|---|---|---|---|---|---|
| (2 < | | Period of Treatment | | | |
| EASI ≦ 10) | Test Group | 2 weeks | 4 weeks | 6 weeks | 8 weeks |
| Female | 0.07% (n = 18) | 0.64 | 1.72 | 2.37 | 2.73 |
| | 0.3% (n = 16) | 0.34 | 0.97 | 1.89 | 2.67 |
| | Control Drug | 0.51 | 0.54 | 0.89 | 1.26 |
| | (n = 16) | | | | |
| Male | 0.07% (N = 12) | 0.08 | 1.03 | 0.90 | 1.34 |
| | 0.3% (N = 16) | 0.41 | 1.09 | 1.19 | 1.51 |
| | Control Drug | 0.94 | 1.02 | 1.45 | 2.27 |
| | (N = 18) | | | | |

TABLE 10
Percentage Change in EASI Score

| Male/Female | | | | | |
|---|---|---|---|---|---|
| (2 < | | Period of Treatment | | | |
| EASI ≦ 10) | Test Group | 2 weeks | 4 weeks | 6 weeks | 8 weeks |
| Female | 0.07% (n = 18) | 13.90 | 30.45 | 42.41 | 48.96 |
| | 0.3% (n = 16) | 7.53 | 19.52 | 30.41 | 42.78 |
| | Control Drug | 6.19 | 9.50 | 14.69 | 23.59 |
| | (n = 16) | | | | |
| Male | 0.07% (N = 12) | 0.43 | 19.35 | 16.38 | 26.60 |
| | 0.3% (N = 16) | 9.69 | 20.92 | 23.95 | 26.35 |
| | Control Drug | 22.09 | 22.30 | 30.30 | 42.58 |
| | (N = 18) | | | | |

TABLE 11
IGA Treatment Success Rate

| Male/Female | | | | | |
|---|---|---|---|---|---|
| (2 < | | Period of Treatment | | | |
| EASI ≦ 10) | Test Group | 2 weeks | 4 weeks | 6 weeks | 8 weeks |
| Female | 0.07% (n = 18) | 5.56 | 11.11 | 16.67 | 50.00 |
| | 0.3% (n = 16) | 0.00 | 6.25 | 12.50 | 31.25 |
| | Control Drug | 12.50 | 6.25 | 18.75 | 25.00 |
| | (n = 16) | | | | |
| Male | 0.07% (N = 12) | 0.00 | 7.69 | 7.69 | 15.38 |
| | 0.3% (N = 16) | 6.25 | 6.25 | 12.50 | 18.75 |
| | Control Drug | 5.56 | 11.11 | 16.67 | 22.22 |
| | (N = 18) | | | | |

TABLE 12
Change in IGA Score

| Male/Female | | | | | |
|---|---|---|---|---|---|
| (2 < | | Period of Treatment | | | |
| EASI ≦ 10) | Test Group | 2 weeks | 4 weeks | 6 weeks | 8 weeks |
| Female | 0.07% (n = 18) | 0.22 | 0.28 | 0.50 | 0.83 |
| | 0.3% (n = 16) | 0.19 | 0.38 | 0.38 | 0.75 |
| | Control Drug | 0.00 | 0.00 | 0.19 | 0.31 |
| | (n = 16) | | | | |
| Male | 0.07% (N = 12) | 0.00 | 0.08 | 0.15 | 0.31 |
| | 0.3% (N = 16) | 0.06 | 0.13 | 0.19 | 0.44 |
| | Control Drug | 0.11 | 0.22 | 0.33 | 0.50 |
| | (N = 18) | | | | |

TABLE 13
Evaluation of Degree of Pruritus

| Male/Female | | | | | |
|---|---|---|---|---|---|
| (2 < | | Period of Treatment | | | |
| EASI ≦ 10) | Test Group | 2 weeks | 4 weeks | 6 weeks | 8 weeks |
| Female | 0.07% (n = 18) | 1.20 | 2.03 | 2.22 | 2.47 |
| | 0.3% (n = 16) | 0.86 | 1.15 | 1.46 | 2.54 |
| | Control Drug | 0.86 | 0.81 | 1.08 | 1.21 |
| | (n = 16) | | | | |
| Male | 0.07% (N = 12) | 1.23 | 2.34 | 1.99 | 2.80 |
| | 0.3% (N = 16) | 1.01 | 1.89 | 2.23 | 2.79 |
| | Control Drug | 1.03 | 1.02 | 1.46 | 2.03 |
| | (N = 18) | | | | |

TABLE 14
Evaluation of Degree of Insomnia

| Male/Female | | | | | |
|---|---|---|---|---|---|
| (2 < | | Period of Treatment | | | |
| EASI ≦ 10) | Test Group | 2 weeks | 4 weeks | 6 weeks | 8 weeks |
| Female | 0.07% (n = 18) | 0.74 | 0.99 | 1.61 | 2.18 |
| | 0.3% (n = 16) | 0.11 | 0.33 | 1.23 | 1.67 |
| | Control Drug | 0.39 | 0.32 | 0.54 | 0.78 |
| | (n = 16) | | | | |
| Male | 0.07% (N = 12) | −0.43 | 0.98 | 0.64 | 1.17 |
| | 0.3% (N = 16) | 1.27 | 1.68 | 1.68 | 2.04 |
| | Control Drug | 0.66 | 0.77 | 0.86 | 1.13 |
| | (N = 18) | | | | |

TABLE 15
Evaluation of Change in Quality of Life

| Male/Female | | | | | |
|---|---|---|---|---|---|
| (2 < | | Period of Treatment | | | |
| EASI ≦ 10) | Test Group | 2 weeks | 4 weeks | 6 weeks | 8 weeks |
| Female | 0.07% (n = 18) | 1.61 | 3.06 | 4.33 | 4.67 |
| | 0.3% (n = 16) | 1.25 | 2.19 | 3.13 | 3.75 |
| | Control Drug | 0.94 | 1.13 | 1.25 | 1.50 |
| | (n = 16) | | | | |
| Male | 0.07% (N = 12) | 1.67 | 3.42 | 3.00 | 5.08 |
| | 0.3% (N = 16) | 1.25 | 2.88 | 3.63 | 4.38 |
| | Control Drug | 1.17 | 1.56 | 1.78 | 2.56 |
| | (N = 18) | | | | |

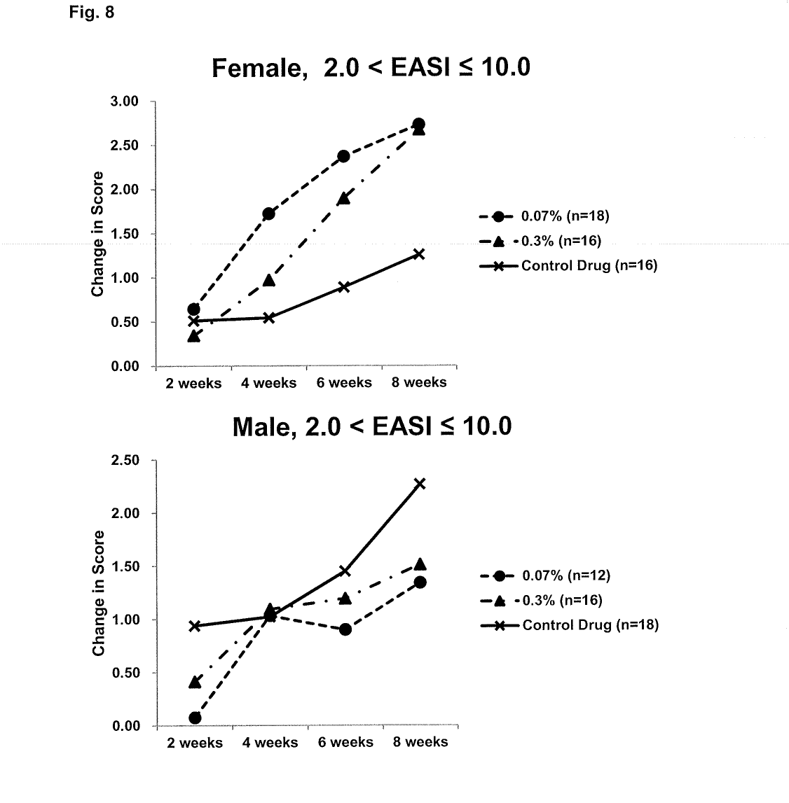

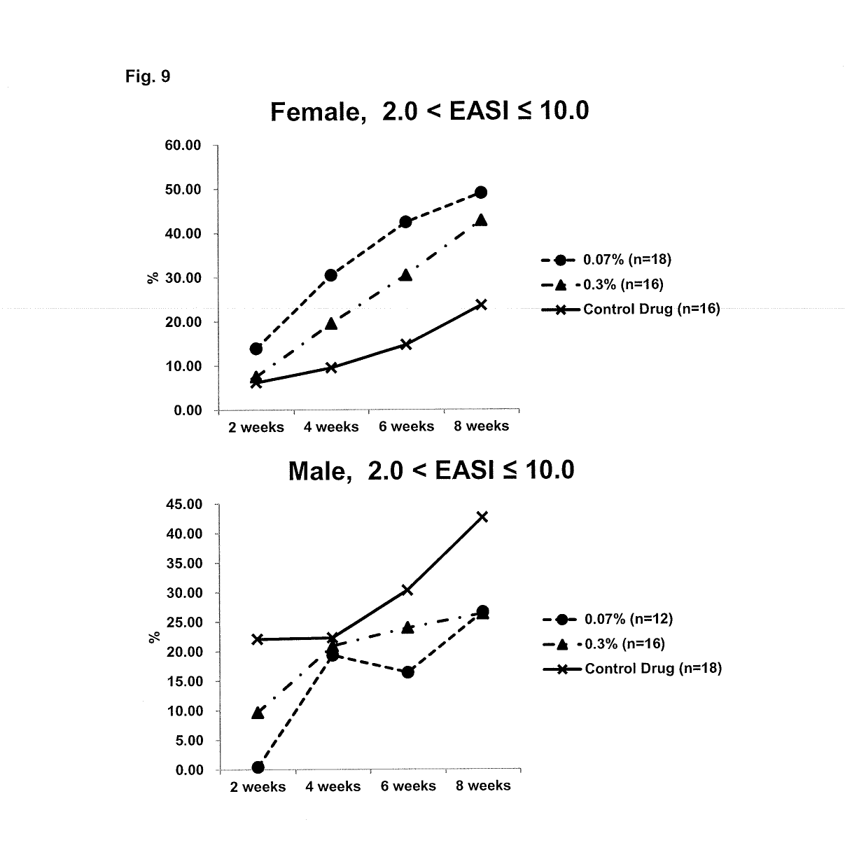

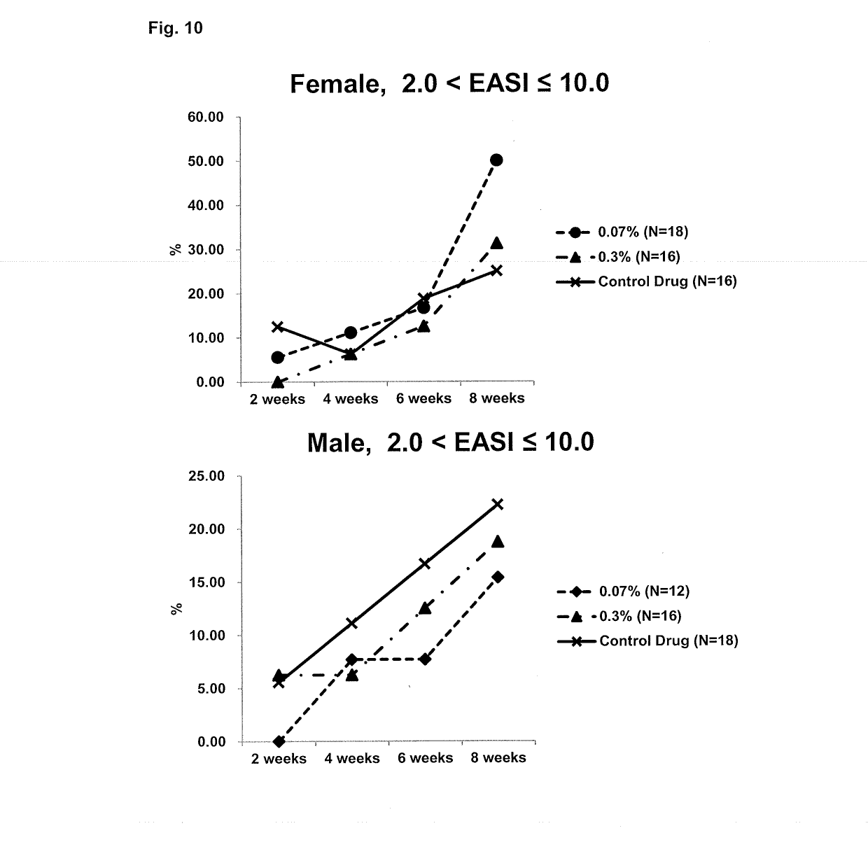

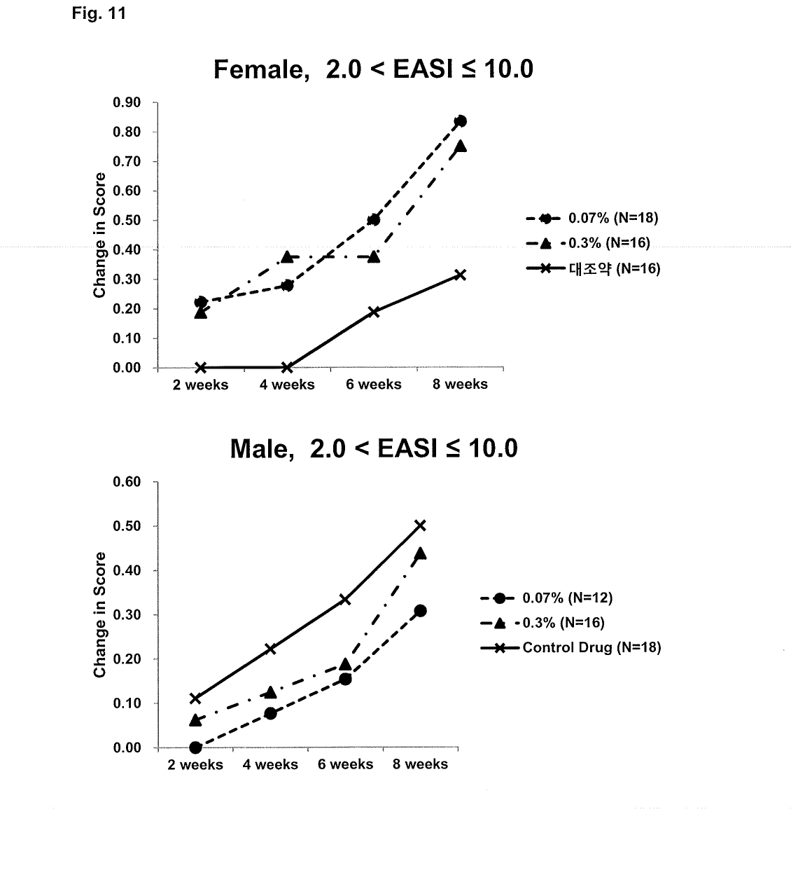

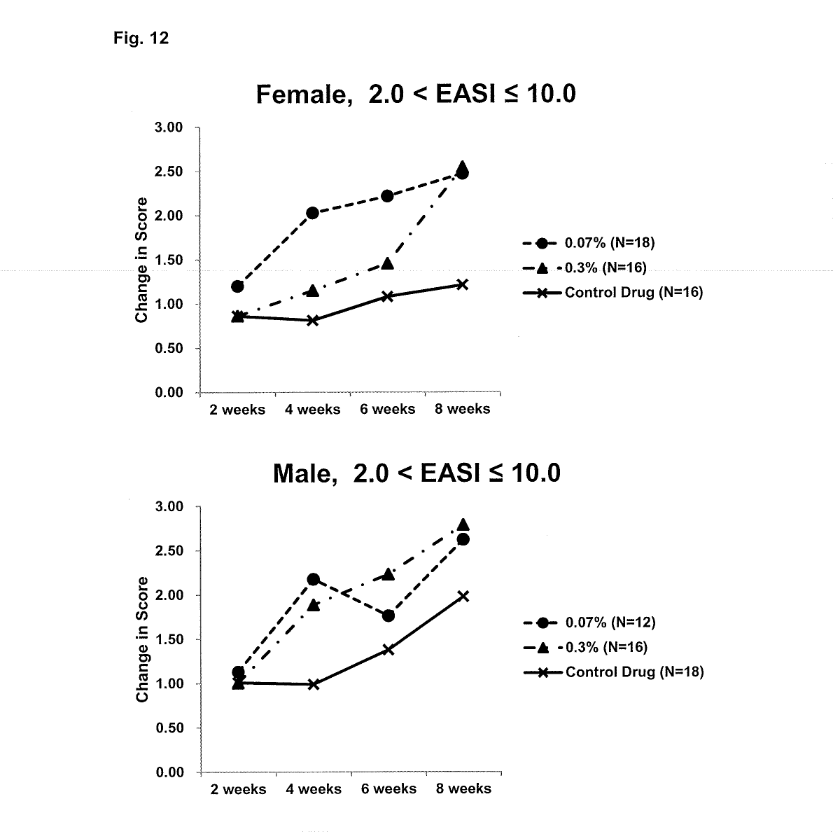

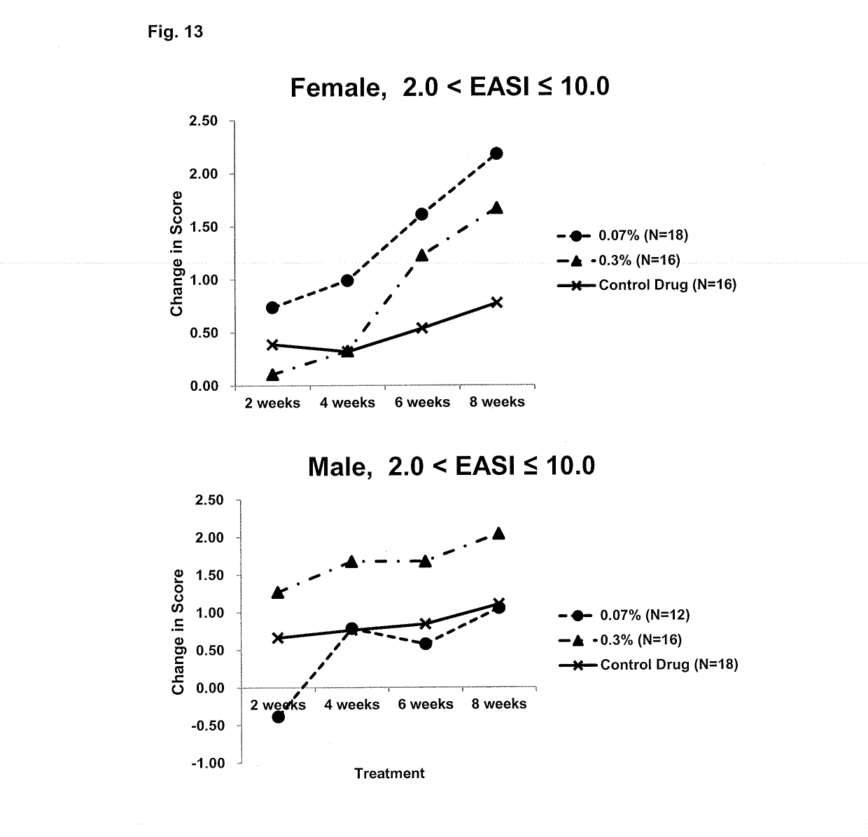

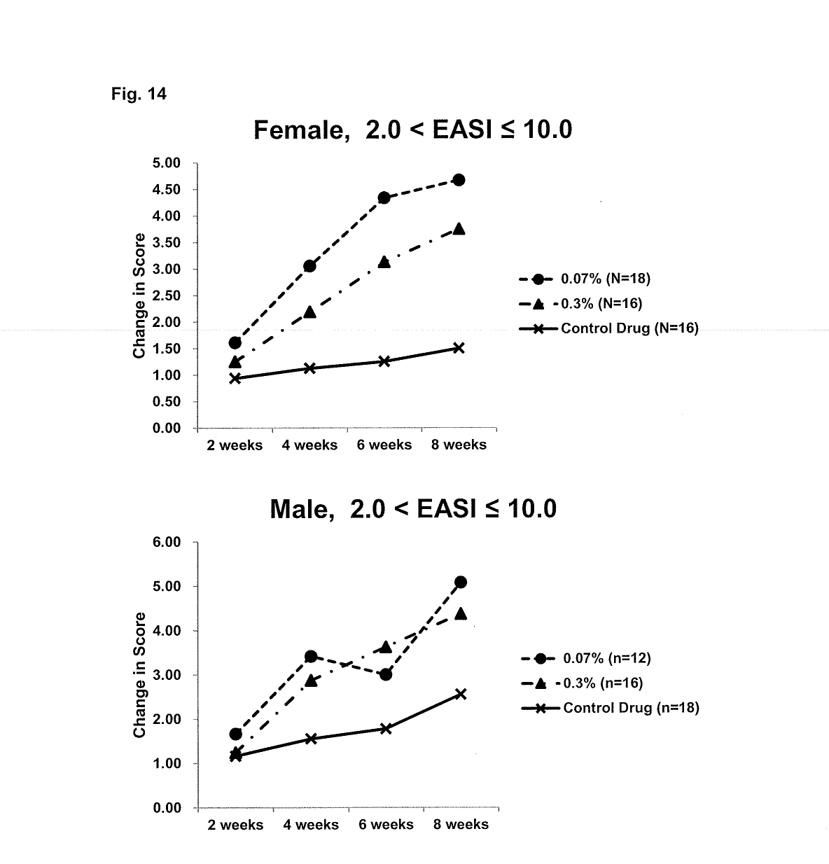

As shown in Tables 9 to 15 and FIGS. 8 to 14, it can be seen that, among mild atopic dermatitis patients having an EASI score of 10 or less but more than 2, adenosylcobalamin was a more effective treatment of atopic dermatitis in female patents than in male patients, in all test categories.

1. An external composition containing adenosylcobalamin as an active ingredient to treat mild atopic dermatitis patients having EASI score of 10 or less but more than 2.

2. An external composition containing adenosylcobalamin as an active ingredient to treat female mild atopic dermatitis patients having EASI score of 10 or less but more than 2.

3. The external composition of claim 1, wherein the composition is formed as a gel, in which the adenosylcobalamin is encapsulated with a liposome.

4. The external composition of claim 1, wherein adenosylcobalamin is contained at 0.01 to 7 wt % based on the total composition of the external composition.

5. A method of treating mild atopic dermatitis patients having EASI score of 10 or less but more than 2, comprising:
locally administering an external composition containing adenosylcobalamin as an active ingredient.

6. A method of treating female mild atopic dermatitis patients having EASI score of 10 or less but more than 2, comprising:
locally administering an external composition containing adenosylcobalamin as an active ingredient.

7. The method of claim 5, wherein the local administration is performed for 4 weeks or more.

8. The method of claim 5, wherein the local administration is performed for 6 weeks or more.

9. The method of claim 5, wherein the local administration is performed for 8 weeks or more.

10. The method of claim 5, wherein the local administration is performed by applying a thin layer of the composition twice a day at intervals of approximately 8 to 14 hours.

11. The external composition of claim 2, wherein the composition is formed as a gel, in which the adenosylcobalamin is encapsulated with a liposome.

12. The external composition of claim 2, wherein adenosylcobalamin is contained at 0.01 to 7 wt % based on the total composition of the external composition.

13. The method of claim 6, wherein the local administration is performed for 4 weeks or more.

14. The method of claim 6, wherein the local administration is performed for 6 weeks or more.

15. The method of claim 6, wherein the local administration is performed for 8 weeks or more.

16. The method of claim 6, wherein the local administration is performed by applying a thin layer of the composition twice a day at intervals of approximately 8 to 14 hours.
